(19) United States
(12) Patent Application Publication
(10) Pub. No.: US 2021/0332318 A1
(43) Pub. Date: Oct. 28, 2021

(54) CELL STRUCTURE FIXING AND REMOVING SYSTEM

(71) Applicant: Cyfuse Biomedical K.K., Tokyo (JP)

(72) Inventor: Yasuto KISHII, Tokyo (JP)

(73) Assignee: Cyfuse Biomedical K.K., Tokyo (JP)

(21) Appl. No.: 16/616,347

(22) PCT Filed: Feb. 1, 2019
(86) PCT No.: PCT/JP2019/003741

§ 371 Date: Nov. 22, 2019

(51) Int. Cl. C12M 1/12; C12M 1/26

(52) U.S. Cl. CPC C12M 25/14; C12M 33/04

(57) ABSTRACT

The cell structure fixing and removing system includes: an alignment base made of a silicone resin, the alignment base including a flat sticking surface to which needles with skewed cell aggregates are to be stuck; and a cell structure removing tool placed on the sticking surface between the alignment base and the cell aggregate, the cell structure removing tool including: a frame extending to correspond to an outline of the sticking surface, the frame including a puncture opening in middle, a bottom surface of the frame forming an alignment surface parallel to the sticking surface; and a porous sheet stretched and arranged on the alignment surface to close the puncture opening of the frame, the porous sheet allowing penetration by the needle, wherein an outline edge of the puncture opening in the alignment surface of the frame of the cell structure removing tool is located in the outline of the sticking surface.

### TECHNICAL FIELD

The present invention relates to a cell structure fixing and removing system for cell structure manufacturing used for manufacturing the three-dimensional structure of cells, and specifically relates to an assembly of a removing tool for cell structure manufacturing and an alignment base.

### BACKGROUND ART

The technique of producing a three-dimensional structure is known that utilizes the character of cell aggregates that the cell aggregates contacting to be adjacent to each other are fused, arranges a plurality of cell aggregates (spheroids) to be adjacent on a plurality of needle-like bodies, and laminates and cultivates these cell aggregates in three dimensions. For example, the technology of producing a three-dimensional structure is disclosed in PTL 1. In this technology, first, a large number of small cells are aggregated into cell aggregates having certain sizes on a cultivation plate, these cell aggregates are transported to respective wells of a laminated tray, one thin needle-like body is moved up and down in the vertical direction for a cell aggregate on any of the respective wells to pierce the cell aggregate, and this is continuously repeated for a plurality of cell aggregates, thereby producing one needle-like body N with a plurality of skewed cell aggregates. Then, as shown in FIG. 7 and FIG. 8, one needle-like body N with a plurality of skewed cell aggregates S is stuck and fixed to an alignment base B. This is repeated for a plurality of needle-like bodies Nm and a plurality of cell aggregates S, and cultivation is performed in the state where the plurality of needle-like bodies Nm with the plurality of skewed cell aggregates are stuck to the alignment base B. The adjacent cell aggregates S are fused to each other by cultivation to form a cell structure Sst having a three-dimensional structure. After the cell structure Sst is formed, when the cell structure Sst is removed from these needle-like bodies Nm, a three-dimensional structure Sst of structurally independent cells will be obtained. The alignment base B to which the needle-like body Nm with skewed cell aggregates S is stuck, and a removing tray for removing the cell structure after cultivation are disclosed in PTL 2. The removing tray F is a jig formed by attaching a porous member Ps in an opening in the middle of a frame having the opening in the middle. When sticking the plurality of needle-like bodies Nm with the plurality of skewed cell aggregates to the alignment base B, the alignment base B and the removing tray F are arranged so that the needle-like bodies Nm penetrate the porous member Ps, and are stuck and fixed to the alignment base B. When removing the cell structure Sst after cultivation from the alignment base B, the removing tray F is moved along the direction in which the needle-like bodies Nm stuck to the alignment base B extend. Then, the porous member Ps of the removing tray F contacts the bottom of the cell structure Sst. When it is continued to move the removing tray F upward along the direction in which the needle-like bodies Nm extend as it is, the porous member Ps pushes up the bottom of the cell structure Sst, and the cell structure Sst can be removed from the alignment base B. For example, the alignment base B has a rectangular parallelepiped shape whose cross-section of a major part is a square. Since the square has a comparatively small shape whose one side is typically 20 millimeters, the removing tray F is grasped and operated by the tips of tweezers.

### CITATION LIST

### Patent Literature

PTL 1: Japanese Patent No. 6334837

PTL 2: Japanese Patent Application Laid-Open No. 2018-143171

### SUMMARY OF INVENTION

### Technical Problem

When removing the cell structure Sst after cultivation from the needle-like bodies Nm, it is necessary to remove in the state where a force is applied to the cell structure Sst only in the direction along the needle-like bodies, and the other load becomes as small as possible, so that the damage to the cell structure Sst becomes small. However, in the removing tray F disclosed in PTL 2, there is a problem that it is difficult to ensure a surface perpendicular to the extending direction of the needle-like bodies while a long time elapses during the cultivation, since the porous member Ps is actually a soft material, and is not stabilized with respect to the alignment base B. Additionally, since the porous member Ps is a porous sheet such as a nonwoven fabric, which is a soft material, and bending actually occurs, when the perpendicular surface cannot be ensured with respect to the extending direction of the needle-like bodies Nm, a force in the direction other than the direction along the needle-like bodies is greatly applied to the cell structure Sst.

### Solution to Problem

A solution is made by a cell structure fixing and removing system including: an alignment base made of a silicone resin, the alignment base including a flat sticking surface to which needles with skewed cell aggregates are to be stuck; and a cell structure removing tool placed on the sticking surface between the alignment base and the cell aggregate, the cell structure removing tool including: a frame extending to correspond to an outline of the sticking surface, the frame including a puncture opening in middle, a bottom surface of the frame forming an alignment surface parallel to the sticking surface; and a porous sheet stretched and arranged on the alignment surface to close the puncture opening of the frame, the porous sheet allowing penetration by the needle, wherein an outline edge of the puncture opening in the alignment surface of the frame of the cell structure removing tool is located in the outline of the sticking surface.

A solution is made by a cell structure removing tool of a cell structure fixing and removing system, the cell structure fixing and removing system including: an alignment base made of a silicone resin, the alignment base including a flat sticking surface to which needles with skewed cell aggregates are to be stuck; and the cell structure removing tool placed on the sticking surface between the alignment base and the cell aggregate, the cell structure removing tool including: a frame extending to correspond to an outline of the sticking surface, the frame including a puncture opening in middle, a bottom surface of the frame forming an alignment surface parallel to the sticking surface; and a porous sheet stretched and arranged on the alignment surface to close the puncture opening of the frame, the porous sheet allowing penetration by the needle, wherein an outline edge of the puncture opening in the alignment surface of the frame of the cell structure removing tool is located in the outline of the sticking surface.

A solution is made by an alignment base of a cell structure fixing and removing system, the cell structure fixing and removing system including: the alignment base made of a silicone resin, the alignment base including a flat sticking surface to which needles with skewed cell aggregates are to be stuck; and a cell structure removing tool placed on the sticking surface between the alignment base and the cell aggregate, the cell structure removing tool including: a frame extending to correspond to an outline of the sticking surface, the frame including a puncture opening in middle, a bottom surface of the frame forming an alignment surface parallel to the sticking surface; and a porous sheet stretched and arranged on the alignment surface to close the puncture opening of the frame, the porous sheet allowing penetration by the needle, wherein an outline edge of the puncture opening in the alignment surface of the frame of the cell structure removing tool is located in the outline of the sticking surface.

### Advantageous Effects of Invention

According to the cell structure fixing and removing system of the present invention, the removing tool can be stabilized with respect to the alignment base.

### BRIEF DESCRIPTION OF DRAWINGS

FIG. 1 is a diagram showing a cell structure fixing and removing system of a first embodiment of the present invention.

FIG. 2 is a diagram of a cell structure fixing and removing system of the first embodiment of the present invention, where a removing tool is separated from an alignment base.

FIG. 3A is a diagram of the removing tool of the first embodiment of the present invention seen from an upper side arrow view 3A of FIG. 1 of the cell structure fixing and removing system.

FIG. 3B is a diagram showing a cross-section 3B-3B of FIG. 1, and is a diagram of the removing tool of first embodiment of the present invention seen from a side surface of the cell structure fixing and removing system.

FIG. 3C is a diagram showing a cross-section 3C-3C of FIG. 2, and is a diagram of the removing tool in the state where it is separated from an alignment base of the first embodiment of the present invention, seen from a side surface of the cell structure fixing and removing system.

FIG. 4 is a diagram showing the cell structure fixing and removing system of a second embodiment of the present invention.

FIG. 5 is a diagram of the cell structure fixing and removing system of the second embodiment of the present invention, where the removing tool is separated from the alignment base.

FIG. 6A is a diagram of the removing tool of the second embodiment of the present invention seen from an upper side arrow view 6A of the cell structure fixing and removing system.

FIG. 6B is a diagram showing a cross-section 6B-6B of FIG. 4, and is a diagram of the removing tool of the second embodiment of the present invention seen from a side surface of the cell structure fixing and removing system.

FIG. 6C is a diagram showing a cross-section 6C-6C of FIG. 5, and is a diagram of the removing tool in the state where it is separated from the alignment base of the second embodiment of the present invention, seen from a side surface of the cell structure fixing and removing system.

FIG. 7 is a diagram showing the concept of fixing needle-like bodies with skewed cell aggregates to the alignment base.

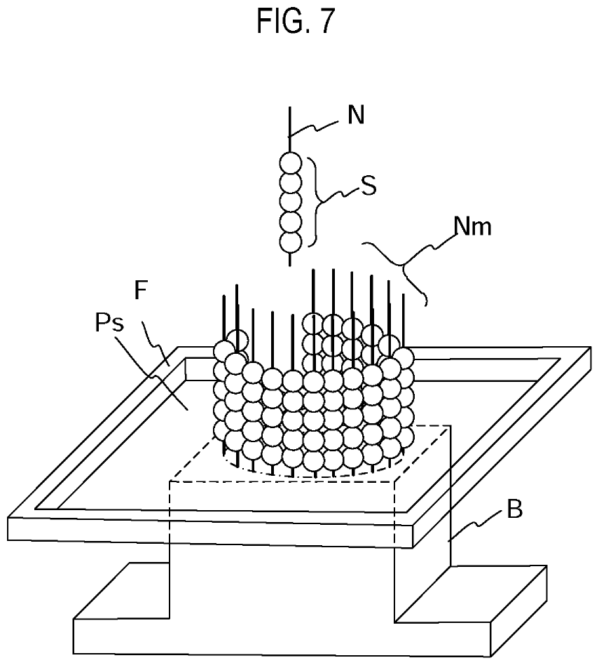

FIG. 8 is a diagram showing the concept of removing a cell structure from the needle-like bodies fixed to the alignment base.

### DESCRIPTION OF EMBODIMENTS

### First Embodiment

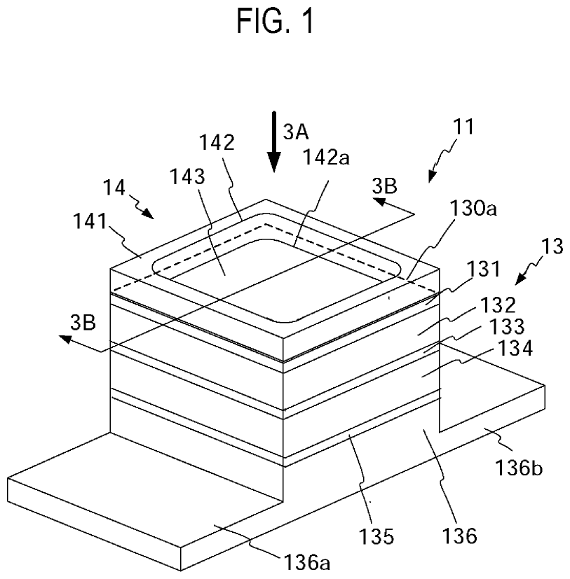

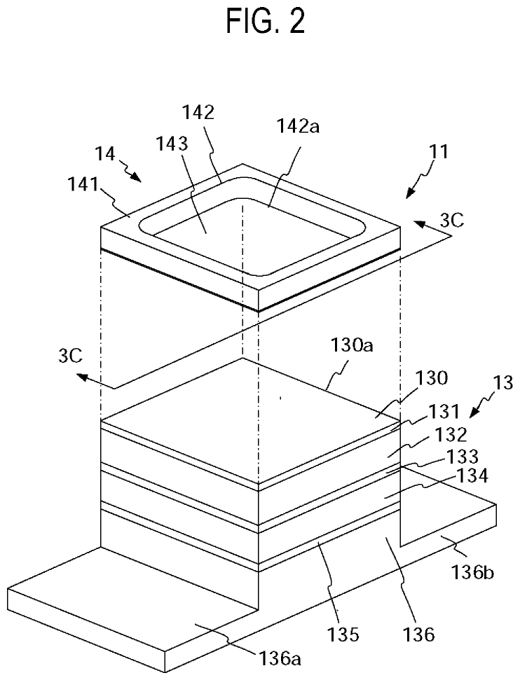

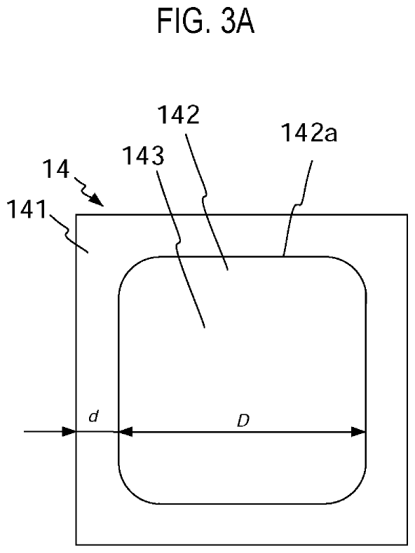

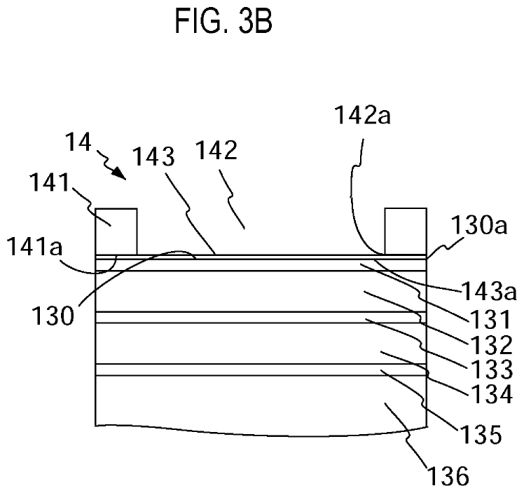

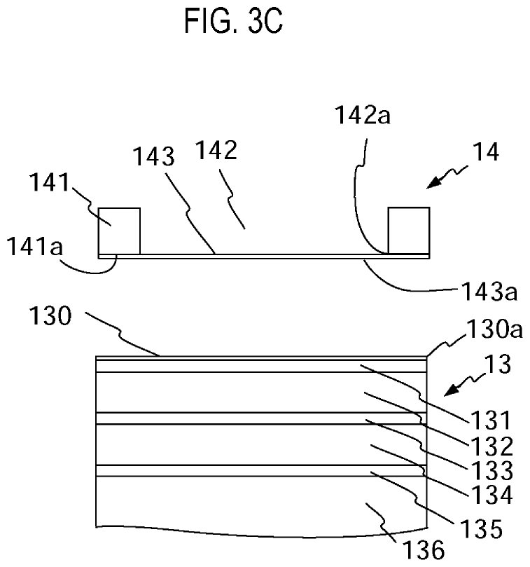

Referring to FIG. 1 to FIG. 3C, a description will be given of a cell structure fixing and removing system 11 of a first embodiment of the present invention. The cell structure fixing and removing system 11 includes an alignment base 13 and a cell structure removing tool 14. FIG. 1 is a perspective view of the cell structure fixing and removing system 11 of the first embodiment of the present invention. FIG. 2 is a perspective view in the state where the cell structure removing tool 14 is separated from the alignment base 13 in the cell structure fixing and removing system 11. FIG. 3A is a diagram seen from an upper side arrow view 3A of FIG. 1. FIG. 3B is a diagram showing a cross-section 3B-3B of FIG. 1, and is a diagram seen from a side surface of the cell structure fixing and removing system 11 in the state where the cell structure removing tool 14 is placed on the alignment base 13. FIG. 3C is a diagram showing a cross-section 3C-3C of FIG. 2, and is a diagram seen from a side surface of the cell structure fixing and removing system 11 in the state where the cell structure removing tool 14 is separated from the alignment base 13.

The alignment base 13 is a self-standable base hardened into a predetermined shape with a silicone resin, and for example, a major part is a rectangular parallelepiped shape having a square cross-section. The square has a comparatively small shape whose one side as typically 20 millimeters. The top face of the alignment base 13 constitutes a sticking surface 130, which as a flat surface that becomes level, when the alignment base 13 is placed in a predetermined state. Needle-like bodies with skewed cell aggregates are stuck to the sticking surface 130 in the vertical direction. That is, the sticking surface 130 extends perpendicularly to the direction in which the needle-like bodies with the skewed cell aggregates are stuck. Although the shape of the sticking surface 130 can be freely set, typically, the shape of the sticking surface 130 is a square shape.

In this description, the direction in which the needle-like bodies are skewed is defined as “the main axis direction”. In the alignment base 13, two silicone resin layers, a first high hardness silicone resin layer 131 and a second high hardness silicone resin layer 133, are formed apart from each other, to extend vertically to the main axis direction. The first high hardness silicone resin layer 131 and the second high hardness silicone resin layer 133 are sheet-like silicone resin. The first high hardness silicone resin layer 131 and the second high hardness silicone resin layer 133 will be formed in parallel with the sticking surface 130. The interlayer distance between the first high hardness silicone resin layer 131 and the second high hardness silicone resin layer 133 is set such that, when needle-like bodies are stuck from the sticking surface 130, if the needle-like bodies are stuck from the sticking surface 130, the needle-like bodies penetrate through the first high hardness silicone resin layer 131, and at least the tips of the needle-like bodies reach the second high hardness silicone resin layer 133.

A gel-like first low hardness silicone resin layer 132 is formed between the first high hardness silicone resin layer 131 and the second high hardness silicone resin layer 133. The three layers, the first high hardness silicone resin layer 131, the first low hardness silicone resin layer 132, and the second high hardness silicone resin layer 133, are formed on a gel-like second low hardness silicone resin layer 134. The respective hardnesses of the first high hardness silicone resin layer 131 and the second high hardness silicone resin layer 133 are higher than the hardness of the first low hardness silicone resin layer 132. Especially, the hardness of the first high hardness silicone resin layer 131 needs to have the hardness that can maintain a flat surface without the first high hardness silicone resin layer 131 striking a wave by the time the needle-like bodies are stuck to the first high hardness silicone resin layer 131 and reach the second high hardness silicone resin layer 133. Then, as for the hardnesses of the first high hardness silicone resin layer 131 and the second high hardness silicone resin layer 133, it is preferable that the needle-like bodies can penetrate, and the needle-like bodies can be maintained at two places, the first high hardness silicone resin layer 131 and the second high hardness silicone resin layer 133. The hardnesses of the first high hardness silicone resin layer 131 and the second high hardness silicone resin layer 133 may be the same, or may be different. When the needle-like bodies are stuck from the sticking surface 130, the needle-like bodies are supported by the first high hardness silicone resin layer 131 and the second high hardness silicone resin layer 133, so that the needle-like bodies extend in the main axis direction. For example, the thickness of the first high hardness silicone resin layer 131 can be 0.5 millimeter, the thickness of the second high hardness silicone resin layer 133 can be 0.2 millimeter, and the thickness of the first low hardness silicone resin layer 132 can be 2 millimeters. These sizes can be arbitrarily set. At least the thickness for the needle-like bodies to reach the second high hardness silicone resin layer 133 from the first high hardness silicone resin layer 131 is set as the thickness for the needle-like bodies to penetrate the second high hardness silicone resin layer 133 from the first high hardness silicone resin layer 131.

Further, in addition to the configurations of the first high hardness silicone resin layer 131 and the second high hardness silicone resin layer 133, a third high hardness silicone resin layer 135 of a sheet-like silicone resin can be arranged apart from the second high hardness silicone resin layer 133. Then, a gel-like second low hardness silicone resin layer 134 may be between the second high hardness silicone resin layer 133 and the third high hardness silicone resin layers 135. For example, although the thickness of the third high hardness silicone resin layer 135 can be 0.5 millimeter, and the thickness of the second low hardness silicone resin layer 134 can be 2 millimeters, the thicknesses can arbitrarily set.

The resin layer group from the first high hardness silicone resin layer 131 having the sticking surface 130 to the third high hardness silicone resin layer 135 is bonded to the upper surface of a stand base 136 with an adhesive. The stand base 136 is a stand formed of, for example, a silicone resin. The bottom surface of the stand base 136 is a flat surface, and can horizontally maintain the first high hardness silicone resin layer 131 to the third high hardness silicone resin layer 135. Ear portions 136a and 136b projecting in the opposite directions are formed on the bottom surface side of the stand base 136. The stand base 136 can be fixed by pressing the ear portions 136a and 136b.

The cell structure removing tool 14 is placed on the sticking surface 130 of the alignment base 13, and the alignment base 13 and the cell structure removing tool 14 are integrally combined in a separable manner. When the needle-like bodies N with the skewed cell aggregates S are stuck to the sticking surface 130, the position of the cell structure removing tool 14 is set so that the porous sheet 143 of the cell structure removing tool 14 is located between the sticking surface 130 and the cell aggregates S.

The cell structure removing tool 14 includes a frame 141, a puncture opening 142, and the porous sheet 143. The frame 141 is a closed frame structure that extends along an outline 130a of the sticking surface 130 with side members corresponding to the respective sides of the outline 130a of the sticking surface 130, when the cell structure removing tool 14 is placed on the sticking surface 130 of the alignment base 13. The puncture opening 142 is formed in a center portion surrounded by the frame 141. The edge of the frame 141 forming the puncture opening 142 forms an outline edge 142a of the puncture opening 142. The surface formed with the outline edge 142a of the puncture opening 142 is perpendicular to the main axis direction. An alignment surface 141a, which is the bottom surface of the frame 141 at the time when the cell structure removing tool 14 is placed on the sticking surface 130, forms an alignment surface parallel to the sticking surface 130. This alignment surface becomes the same surface as the surface formed by the outline edge 142a of the puncture opening 142.

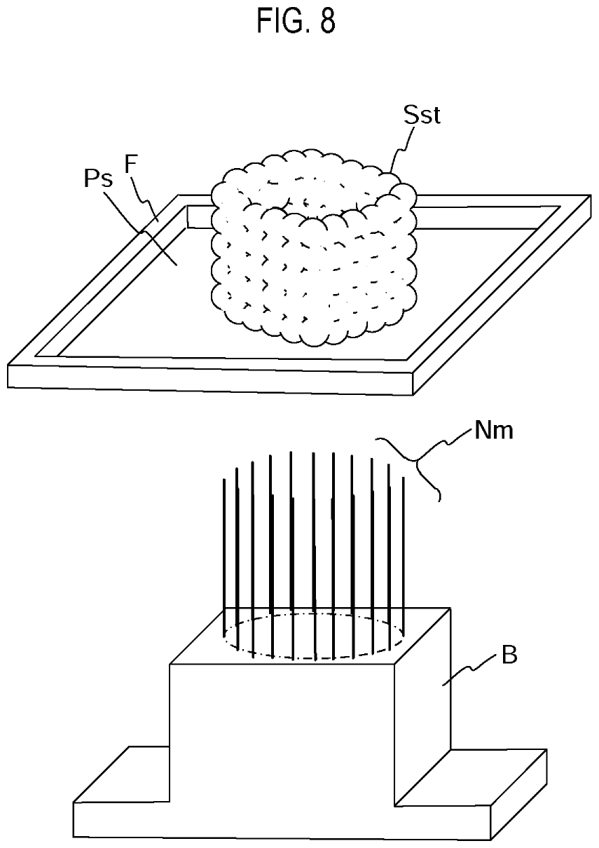

The porous sheet 143 is attached to the alignment surface 141a of the frame 141 in a stretched manner to adhere thereto and not to be relaxed, so that the whole surface of the surface formed by the outline edge 142a of the puncture opening 142 is covered to close the puncture opening 142. The porous sheet 143 is a sheet-like member having a mesh smaller than the diameter of the needle-like body. For example, the porous sheet 143 is a nonwoven fabric. When the cell structure removing tool 14 is placed on the sticking surface 130 of the alignment base 13, the outline edge 142a of the puncture opening 142 in the alignment surface 141a of the frame 141 will be located inside the outline 130a of the sticking surface 130. Accordingly, the alignment surface 141a of the frame 141 closely adheres with its own weight to press the porous sheet 143 against the sticking surface 130, and the cell structure removing tool 14 can be placed on the sticking surface 130 of the alignment base 13 in the state where the porous sheet 143 is in surface contact with the sticking surface 130. The outer outline of the frame 141 can be made into the same shape as the sticking surface 130. However, by making the outer outline of the frame 141 into the same shape as the outline of the sticking surface 130 of the alignment base 13, there is an effect to make it easier to perform adjustment so that the tips of tweezers correctly and horizontally sandwiches only the cell structure removing tool 14, while sandwiching the alignment base 13 and the cell structure removing tool 14 at the same time, when sandwiching only the cell structure removing tool 14 in order to operate the cell structure removing tool 14 with the tips of the tweezers. Especially, since the alignment base 13 and the cell structure removing tool 14 are small, it is a very significant effect that only the cell structure removing tool 14 can be horizontally and correctly sandwiched, by making the outer outline of the frame 141 into the same shape as the outline of the sticking surface 130 of the alignment base 13, in order to horizontally and correctly sandwich only the cell structure removing tool 14. It is preferable that a width d of the frame 141 with which the alignment surface 141a of the frame 141 contacts the sticking surface 130 via the porous sheet 143 is equal to or more than ⅙ of a cross width D of the puncture opening 142 in the same direction. At this time, the porous sheet 143 of the cell structure removing tool 14 is also attached to adhere to the lower side of the alignment surface 141a by securing at least the width d at the lower side of the cell structure removing tool 14 (the sticking surface 130 side of the first high hardness silicone resin layer 131). When the porous sheet 143 of the cell structure removing tool 14 contacts the sticking surface 130 of the first high hardness silicone resin layer 131, the porous sheet 143 of the cell structure removing tool 14 and the sticking surface 130 of the first high hardness silicone resin layer 131 closely adhere to each other with the own weight of the cell structure removing tool 14. As shown in FIG. 8, when removing the cell structure from the needle-like bodies, only the cell structure removing tool 14 is sandwiched by the tips of tweezers, and lifted to be separated from the alignment base 13. At this time, for example, two tweezers can be used, and the stand base 136 can be pressed downward with the ear portions 136a and 136b of the stand base 136 of the alignment base 13 by the tips of one tweezers (not shown), and only the cell structure removing tool 14 can be sandwiched by the other tweezers (not shown) to be separated from the alignment base 13. Accordingly, the porous sheet 143 of the cell structure removing tool 14 closely adhering to the sticking surface 130 of the first high hardness silicone resin layer 131 can be easily separated from the sticking surface 130 of the first high hardness silicone resin layer 131, and a process of removing the cell structure can be realized in a more stable manner.

By making a surface of the first high hardness silicone resin layer 131 of the alignment base 13 serve as the sticking surface 130, also when the cell structure removing tool 14 is placed on the sticking surface 130, while it becomes easier for the porous sheet 143 to closely adhere, the cell structure removing tool 14 can be easily peeled from the sticking surface 130. Accordingly, when removing a cell structure by the cell structure removing tool 14 after cultivation of cell aggregates ends, by peeling the frame 141 from the sticking surface 130, the porous sheet 143 is separated from the sticking surface 130 without large deformation, and it becomes possible to remove the cell structure from the needle-like bodies, without applying load to the cell structure.

### Second Embodiment

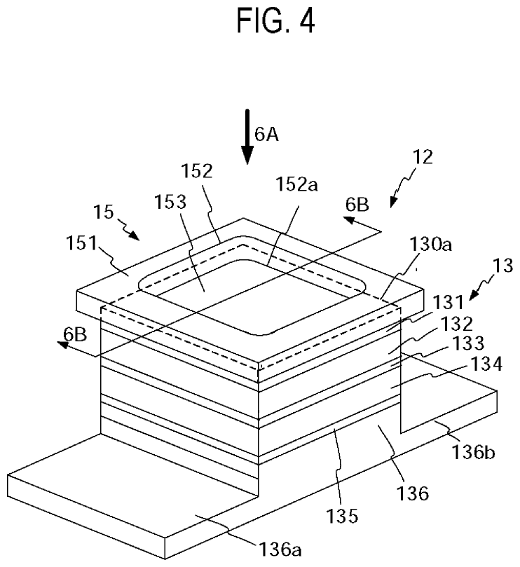

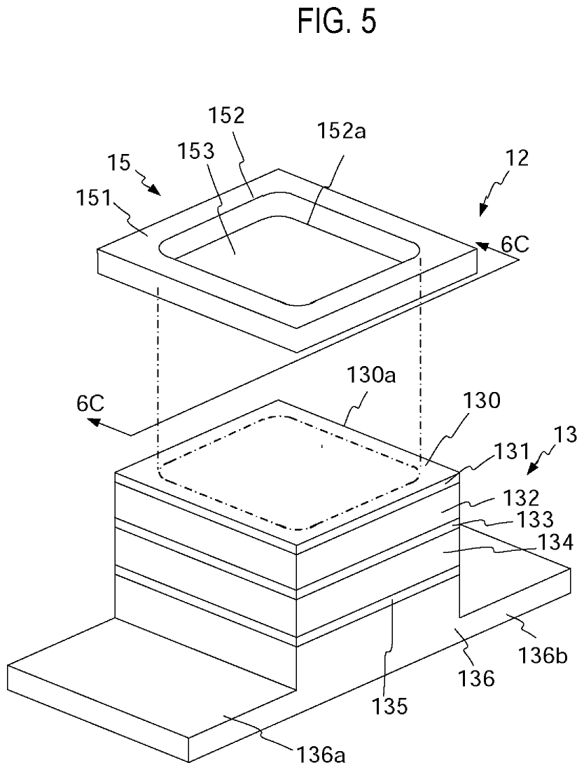

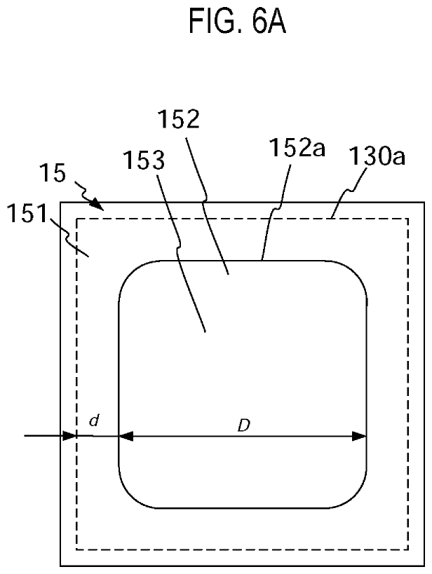

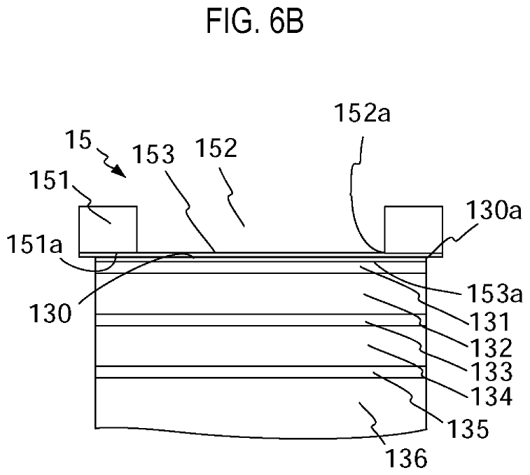

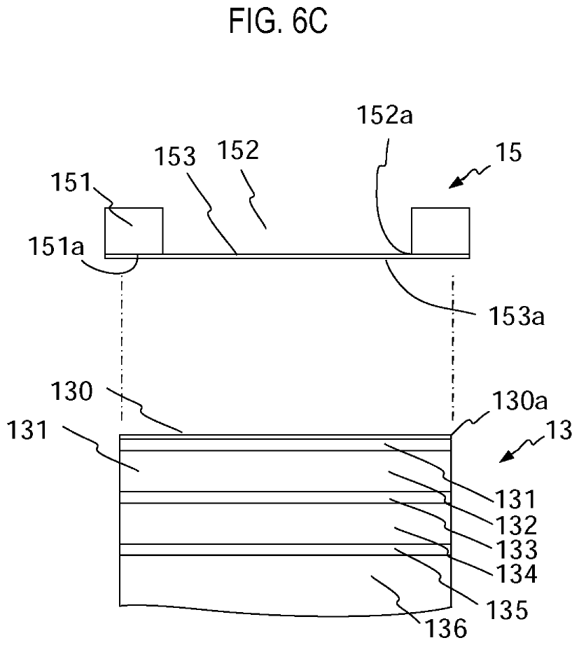

Subsequently, referring to FIG. 4 to FIG. 6, a description will be given of a cell structure fixing and removing system 12 of a second embodiment of the present invention. The cell structure fixing and removing system 12 includes the alignment base 13 and a cell structure removing tool 15. FIG. 4 is a perspective view of the cell structure fixing and removing system 12 of the second embodiment of the present invention. FIG. 5 is a perspective view in the state where the cell structure removing tool 15 is separated from the alignment base 13 in the cell structure fixing and removing system 12. FIG. 6A is a diagram seen from an upper side arrow view 6A of FIG. 4. FIG. 6B is a diagram showing a cross-section 6B-6B of FIG. 4, and is a diagram seen from a side surface of the cell structure fixing and removing system 12 in the state where the cell structure removing tool 15 is placed on the alignment base 13. FIG. 6C is a diagram showing a cross-section 6C-6C of FIG. 5, and is a diagram seen from a side surface of the cell structure fixing and removing system 12 in the state where the cell structure removing tool 15 is separated from the alignment base 13. In the second embodiment, the size of the frame of the cell structure removing tool 15 is different.

The cell structure removing tool 15 includes a frame 151, a puncture opening 152, and a porous sheet 153. The frame 151 is a closed frame structure that extends along the outline 130a of the sticking surface 130 with side members corresponding to the respective sides of the outline 130a of the sticking surface 130, when the cell structure removing tool 15 is placed on the sticking surface 130 of the alignment base 13. The alignment base 13 is completely the same as that in the first embodiment. Hereinafter, only different parts are described here, and the other parts are the same as those in the first embodiment.

Although the outer outline of the frame 141 in the cell structure removing tool 14 in the first embodiment has the same shape as the sticking surface 130, the outer outline of the frame 151 in the cell structure removing tool 15 in the second embodiment are different in that it has a larger shape than the sticking surface 130.

However, the inner outline of the frame 151 is a closed frame structure that extends along the outline 130a of the sticking surface 130 with the side members corresponding to the respective sides of the outline 130a of the sticking surface 130, when the cell structure removing tool 15 is placed on the sticking surface 130 of the alignment base 13, and is the same as that in the first embodiment. Similar to the first embodiment, the porous sheet 153 is also attached to an alignment surface 151a of the frame 151 in a stretched manner to adhere thereto and not to be relaxed, so that the whole surface of the surface formed by an outline edge 152a of the puncture opening 152 is covered to close the puncture opening 152. At this time, when the cell structure removing tool 15 is placed on the sticking surface 130 of the alignment base 13, the porous sheet 153 is secured so that the outline edge 152a of the puncture opening 152 in the alignment surface 151a of the frame 151 is located inside the outline 130a of the sticking surface 130. Accordingly, the alignment surface 151a of the frame 151 closely adheres with its own weight to press the porous sheet 153 against the sticking surface 130, and the cell structure removing tool 15 can be placed on the sticking surface 130 of the alignment base 13 in the state where the porous sheet 153 is in surface contact with the sticking surface 130.

Also in the second embodiment, the puncture opening 152 is formed in the center portion surrounded by the frame 151. The edge of the frame 151 forming the puncture opening 152 forms the outline edge 152a of the puncture opening 152. The surface formed by the outline edge 152a of the puncture opening 152 is perpendicular to the main axis direction. Also in this case, it is preferable that a width d of the frame 151 with which the alignment surface 151a of the frame 151 contacts the sticking surface 130 via the porous sheet 153 is equal to or more than ⅙ of a cross width D of the puncture opening 152 in the same direction. The porous sheet 153 is also attached to adhere to the alignment surface 151a by securing at least the width d.

### REFERENCE SIGNS LIST

11, 12 cell structure fixing and removing system
13 alignment base
14, 15 removing tool
130 sticking surface
131 first high hardness silicone resin layer
132 first low hardness silicone resin layer
133 second high hardness silicone resin layer
134 second low hardness silicone resin layer
141, 151 frame
141a, 151a alignment surface
142, 152 puncture opening
142a, 152a outline edge
143, 153 porous sheet

1. A cell structure fixing and removing system comprising:
an alignment base made of a silicone resin, the alignment base including a flat sticking surface to which needles with skewed cell aggregates are to be stuck; and
a cell structure removing tool placed on the sticking surface between the alignment base and the cell aggregate,
the cell structure removing tool including:
a frame extending to correspond to an outline of the sticking surface, the frame including a puncture opening in middle, a bottom surface of the frame forming an alignment surface parallel to the sticking surface; and
a porous sheet stretched and arranged on the alignment surface to close the puncture opening of the frame, the porous sheet allowing penetration by the needle,
wherein an outline edge of the puncture opening in the alignment surface of the frame of the cell structure removing tool is located in the outline of the sticking surface.

2. A cell structure fixing and removing system according to claim 1,
wherein an outer outline of the frame is a same as the outline of the sticking surface of the alignment base.

3. A cell structure fixing and removing system according to claim 1,
wherein an outer outline of the frame is located outside of the outline of the sticking surface of the alignment base.

4. A cell structure fixing and removing system according to claim 1,
wherein the alignment base is formed to be parallel to the sticking surface, the alignment base including at least two silicone resin layers having a higher hardness than other parts.

5. A cell structure removing tool of a cell structure fixing and removing system, the cell structure fixing and removing system comprising:
an alignment base made of a silicone resin, the alignment base including a flat sticking surface to which needles with skewed cell aggregates are to be stuck; and
the cell structure removing tool placed on the sticking surface between the alignment base and the cell aggregate,
the cell structure removing tool including:
a frame extending to correspond to an outline of the sticking surface, the frame including a puncture opening in middle, a bottom surface of the frame forming an alignment surface parallel to the sticking surface; and
a porous sheet stretched and arranged on the alignment surface to close the puncture opening of the frame, the porous sheet allowing penetration by the needle,
wherein an outline edge of the puncture opening in the alignment surface of the frame of the cell structure removing tool is located in the outline of the sticking surface.

6. A cell structure removing tool according to claim 5,
wherein an outer outline of the frame is a same as the outline of the sticking surface of the alignment base.

7. A cell structure removing tool according to claim 5,
wherein an outer outline of the frame is located outside of the outline of the sticking surface of the alignment base.

8. A cell structure removing tool according to claim 5,
wherein the alignment base is formed to be parallel to the sticking surface, the alignment base including at least two silicone resin layers having a higher hardness than other parts.

9. An alignment base of a cell structure fixing and removing system, the cell structure fixing and removing system comprising:
the alignment base made of a silicone resin, the alignment base including a flat sticking surface to which needles with skewed cell aggregates are to be stuck; and
a cell structure removing tool placed on the sticking surface between the alignment base and the cell aggregate,
the cell structure removing tool including:
a frame extending to correspond to an outline of the sticking surface, the frame including a puncture opening in middle, a bottom surface of the frame forming an alignment surface parallel to the sticking surface; and
a porous sheet stretched and arranged on the alignment surface to close the puncture opening of the frame, the porous sheet allowing penetration by the needle,
wherein an outline edge of the puncture opening in the alignment surface of the frame of the cell structure removing tool is located in the outline of the sticking surface.

10. An alignment base according to claim 9,
wherein an outer outline of the frame is a same as the outline of the sticking surface of the alignment base.

11. An alignment base according to claim 9,
wherein an outer outline of the frame is located outside of the outline of the sticking surface of the alignment base.

12. An alignment base according to claim 9,
wherein the alignment base is formed to be parallel to the sticking surface, the alignment base including at least two silicone resin layers having a higher hardness than other parts.
